SEQUENCE LISTING (27 sequences)

SEQ ID NO: 1 - protein, 7 aa, Homo sapiens
VVLVTSS

SEQ ID NO: 2 - protein, 9 aa, Homo sapiens
CSTSMLKAC

SEQ ID NO: 3 - protein, 9 aa, Homo sapiens
CLIDLHVMC

SEQ ID NO: 4 - protein, 10 aa, Homo sapiens
CTTHWGFTLC

SEQ ID NO: 5 - protein, 28 aa, Homo sapiens
SLRRSSCFGGRMDRIGAQSGLGCNSFRY

SEQ ID NO: 6 - protein, 12 aa, Homo sapiens
CRSWNKADNRSC

SEQ ID NO: 7 - protein, 8 aa, Homo sapiens
DRVYIHPF

SEQ ID NO: 8 - protein, 5 aa, Homo sapiens
CRPPR

SEQ ID NO: 9 - protein, 7 aa, Homo sapiens
QAQGQLV

SEQ ID NO: 10 - protein, 7 aa, Homo sapiens
ARRGQAV

SEQ ID NO: 11 - protein, 7 aa, Homo sapiens
GRRFIRV

SEQ ID NO: 12 - protein, 7 aa, Homo sapiens
VHPKQHR

SEQ ID NO: 13 - protein, 7 aa, Homo sapiens
VHSPNKK

SEQ ID NO: 14 - protein, 9 aa, Homo sapiens
CNNSKSHTC

SEQ ID NO: 15 - protein, 17 aa, Homo sapiens
NNQKIVNLKEKVAQLEA

SEQ ID NO: 16 - protein, 11 aa, Homo sapiens
GPXRSGGGGKC

SEQ ID NO: 17 - protein, 9 aa, Homo sapiens
KKLVPRGSL

SEQ ID NO: 18 - protein, 9 aa, Homo sapiens
CRKRLDRNC

SEQ ID NO: 19 - protein, 9 aa, Homo sapiens
CRTLTVRKC

SEQ ID NO: 20 - protein, 6 aa, Homo sapiens
CKRAVR

SEQ ID NO: 21 - protein, 9 aa, Homo sapiens
CPKTRRVPC

SEQ ID NO: 22 - protein, 6 aa, Homo sapiens
CARPAR

SEQ ID NO: 23 - protein, 9 aa, Homo sapiens
CRSTRANPC

SEQ ID NO: 24 - protein, 7 aa, Homo sapiens
GGGVFWQ

SEQ ID NO: 25 - protein, 7 aa, Homo sapiens
HGRVRPH

SEQ ID NO: 26 - protein, 8 aa, Homo sapiens
CLHRGNSC

SEQ ID NO: 27 - protein, 12 aa, Homo sapiens
CRSWNKADNRSC

(19) United States
(12) Patent Application Publication
(10) Pub. No.: US 2025/0090683 A1
(43) Pub. Date: Mar. 20, 2025

(54) CONJUGATE TARGETING CARDIOVASCULAR DISEASE

(71) Applicant: Suntec Medical, Inc., Walnut, CA (US)

(72) Inventors: Chun-Ting Cheng, Taipei (TW); Yuan-Chung Tsai, Taipei (TW); Pauline Ying Lau, Walnut, CA (US)

(21) Appl. No.: 18/968,852

(22) Filed: Dec. 4, 2024

Related U.S. Application Data
(63) Continuation of application No. PCT/US2023/067917, filed on Jun. 5, 2023.
(60) Provisional application No. 63/349,482, filed on Jun. 6, 2022.

(51) Int. Cl. A61K 47/69; A61K 31/704; A61K 47/54; A61K 47/59; A61K 47/60; A61K 47/61; A61K 47/62; A61P 9/10; C07K 16/28

(52) U.S. Cl. CPC A61K 47/6925; A61K 31/704; A61K 47/545; A61K 47/593; A61K 47/60; A61K 47/61; A61K 47/62; A61P 9/10; C07K 16/2809

(57) ABSTRACT

The present invention provides a conjugate comprising: (a) a cardiovascular disease (CVD)-targeting ligand, (b) a hydrophilic polymer of polyethylene glycol (PEG), polylactic acid (PLA), polylactic-co-glycolic acid (PLGA), or dextran, and (c) a flavonoid. The present invention also provides to a micelle nanoparticle composition comprising: (a) an outer shell comprising the conjugate, optionally (b) an inner shell comprising oligomeric (−)-epigallocatechin gallate (OEGCG), and optionally (c) a CVD-treating molecule encapsulated in the inner shell. The present invention further provides a method for treating a CVD by administering an effective amount of the present nanoparticle composition to a subject. The CVD-targeting ligand targets the heart tissue and delivers active ingredients to heart tissue for treating cardiovascular diseases or conditions.

### CROSS-REFERENCE TO RELATED APPLICATION(S)

This application is a continuation of PCT/US2023/067917, filed Jun. 5, 2023; which claims the benefit of U.S. Provisional Application No. 63/349,482, filed Jun. 6, 2022. The contents of the above-identified applications are incorporated herein by reference in their entireties.

### REFERENCE TO SEQUENCE LISTING, TABLE OR COMPUTER PROGRAM

This application contains an ST.26 compliant Sequence Listing, which is submitted concurrently in xml format via Patent Center and is hereby incorporated by reference in its entirety. The .xml copy, created on Jun. 2, 2023, is named SequenceListing 8006 and is 236000 kilobytes in size.

### FIELD OF THE INVENTION

The present invention relates to a conjugate comprising: (a) a CVD (cardiovascular disease) targeting ligand, (b) a hydrophilic polymer of polyethylene glycol (PEG), polylactic acid (PLA), polylactic-co-glycolic acid (PLGA), or dextran, and (c) a flavonoid, wherein the hydrophilic polymer covalently binds to the flavonoid and the CVD-targeting ligand. The present invention relates to micelle nanoparticles comprising: (a) an outer shell comprising a CVD-targeting ligand-hydrophilic polymer-epigallocatechin gallate (EGCG) conjugate, optionally (b) inner shell comprising oligomeric flavonoid, and optionally (c) a CVD treating agent encapsulated in the inner shell.

### BACKGROUND OF THE INVENTION

Cardiovascular disease (CVD) is a class of diseases that involve the heart or blood vessels. CVD includes coronary artery diseases (e.g. angina and myocardial infarction), stroke, heart failure, hypertensive heart disease, rheumatic heart disease, cardiomyopathy, abnormal heart rhythms, congenital heart disease, valvular heart disease, carditis, aortic aneurysms, peripheral artery disease, thromboembolic disease, and venous thrombosis. Coronary artery disease (CAD) and stroke account for 80% of CVD deaths in males and 75% of CVD deaths in females.

CVD includes hypertension, atherosclerosis, acute myocardial infarction (AMI), heart failure, aortic aneurysm, and restenosis.

Hypertension (high blood pressure) is responsible for the high ratio of mortality contributed by CVDs worldwide. Hypertension is responsible for disease status of significant organs in the body such as heart, brain, blood vessels, eyes, and kidneys. Hypertension also leads to the occurrence of different CVDs, such as ischemia, atherosclerosis, congestive heart failure, and cardiac arrest.

Atherosclerosis is an essential factor of stroke and other CVDs. Retention of lipoproteins on the sub-endothelial extracellular matrix induces the formation of atherosclerotic plaques. The rupture of the plaque can lead to myocardial infarction, and stroke.

AMI and ischemic death of cardiomyocytes are one of the most severe types of atherosclerotic cardiovascular disease.

Heart failure is a complex pathophysiological syndrome that arises due to the impaired function of the heart to fill or eject the blood. Different clinical manifestations have associated with heart failure along with myocardial insults such as genetic factors, hypertension, hypertrophy, and coronary artery disease.

Aortic aneurysm can be dangerous and causes death in case of rupture or dissection. Pathophysiology of aortic aneurysm includes the loss of smooth muscle cells in the aortic wall, chronic inflammation, and destructive connective tissue remodeling

Restenosis occurs as a serious complication of vascular interventional procedures, resulting in abnormal narrowing of the blood vessel. It is because vascular interventional procedures tend to focus on the restoration of blood flow across the obstructed arteries.

Cardiovascular disease (CVD) produces immense health and economic burdens in the United States and globally. According to the American National Health and Nutrition Examination Survey data, there were 121.5 million CVD patients in 2016, about half of the adults suffered from CVD, and the prevalence rate increased with age. In 2016, approximately 17.6 million people worldwide died of CVD, an increase of 14.5% over a decade earlier. Cardiovascular disease is the leading cause of death for people all over the world. It is estimated that 23.6 million people will die of CVD in 2030. All kinds of cardiovascular diseases will develop into heart failure (HF) in the late stage, at which time; conventional treatment methods have a limited therapeutic effect on heart failure.

CVD is a progressive disease cascade originated from the risk factors including dyslipidemia, hypertension, diabetes, smoking and obesity initially, lead to atherosclerosis (a block of blood flow in the arteries) and left ventricular hypertrophy (LVH) followed by coronary artery disease (CAD), myocardial ischemia, coronary thrombosis and myocardial infarction (AMI). At the stage of AMI, significant heart cell damages lead to arrhythmias and loss of muscle which may lead to sudden cardiac death. After initial AMI, the heart begins remodeling. The CVD patients often develops ventricular enlargement, progressively resulted in congested heart failure (CHF), and eventually enter end-stage heart disease. Without an effort to stop CVD progression or seek to promote cardiovascular regeneration, CVD is destined to be a life-threatening disease (Circulation. 2006; 114:2850-2870).

CAD is a major CVD and is the number one cause of death among all the diseases. CAD is a result of the atherosclerosis of the coronary arteries. Atherosclerosis is caused by the accumulation of lipoprotein droplets (Low-density lipoproteins, LDL) in the intima of the coronary vessels with disrupted endothelial function. LDL in high concentration possess the ability to permeate the disrupted endothelium (vasculature cells) and undergo oxidation to attract leukocytes (immune cells including T cell, B cell and more), which are scavenged by macrophages, leading to the formation of foamy-textured cells. These foamy cells replicate and form lesions, which attract smooth muscle cells (SMCs). The SMCs proliferate and produce extracellular matrix of collagen and proteoglycans which leads to fibrous plaque in the lesion. The atherosclerotic plaque in the coronary artery reduces oxygen and nutrient supply to the heart cells and resulted into the death of billions of heart muscle cells (cardiomyocytes) and vasculature (endothelial cells, pericytes). Eventually, the dead cardiovascular tissue is replaced by fibroblast-mediated scar formation, which is non-functional, and results in pathological remodeling of the heart and the disfunction of the conduction system cells (AV node, sinoatrial node, purkinje fibres), and nervous system cells (glial cells, neurons). Taken together, beginning from CAD/atherosclerosis, multiple dysfunctional cells and environmental factors (e.g. LDL, MMP), the progression of CVD ultimately lead to heart failure (J Cell Physiol. 2019; 1-12).

Acute myocardial infarction (AMI) is an ischemic heart disease, caused by coronary artery obstruction. Traditional clinical approaches for myocardial infarction rely thrombolytic agents such as TPA, or on surgical revascularization procedures, such as coronary stenting or coronary artery bypass grafts. AMI can cause massive cardiomyocyte damage, novel therapeutics using cells (especially stem cells), genes, exosomes, and growth factors are emerging and have shown significant research outcomes for cardiomyocyte repair, numerous challenges still exist in translating those technologies into clinical practice.

Myocardium, like other parenchymal. organs, contains endogenous stem cells with the ability to proliferate and replace cardiomyocytes that die due to apoptosis or oncosis. However, the regenerative capacity of the adult human heart is limited and insufficient to overcome the massive loss (>1 billion) of cardiomyocytes during acute damage or prolonged remodeling. Cardiovascular regenerative medicine (CRM) can provide growth factors and cytokines to promote the regeneration of cardiovascular cell regeneration (Eur Heart J. 2017 Sep. 1; 38 (33): 2532-2546.). These CRM can be used in acute myocardial infarction (AMI), chronic ischemic cardiomyopathy (CIC), on dilated cardiomyopathy (DCM), other forms of non-ischaemic heart disease (NIHD), and possibly in other cardiac conditions (e.g. valvular heart disease, rhythm disorders, and congenital myopathies).

CRM products intended to be used on the modulation, enhancement and activation of endogenous regenerative responses can be subdivided into three main groups: (1) Cell implantation; (2) Injection of biological or synthetic factors with active functions in endogenous regenerative processes; (3) Genetic and epigenetic modifications that modulate the expression of genes and mRNA involved in the endogenous regenerative capacity.

Challenges of clinical CRM include:
A combination therapy is required because efforts should show the efficacy in multiple cell types (cardiomyocytes, vascular endothelial cells, fibroblasts) in heart and also the extracellular matrix environments, the electromechanical coupling requirements for a well-coordinated improvement in cardiac contractility. MINC is a platform that can deliver combination therapies to the damaged cardiovascular lesions.
A good delivery system is required to provide sufficient CRM dosages of the treatment drugs to the region of interest of the host tissue to achieve bioavailability and minimal off-target effects.

For many therapeutic agents, only a small portion of the medication reaches the tissue to be affected, for example, in chemotherapy where roughly 99% of the drugs administered do not reach the tumor site. Targeted drug delivery seeks to concentrate the medication in the tissues of interest while reducing the relative concentration of the medication in the remaining tissues. For example, by avoiding the host's defense mechanisms and inhibiting non-specific distribution in the liver and spleen, a system can reach the intended site of action in higher concentrations. Targeted delivery may improve efficacy while reducing side-effects. Cardiac delivery system is designed to specifically deliver the drug/biomolecule to CVD lesions and enhance their accumulation for higher overall efficacy and safety.

### BRIEF DESCRIPTION OF THE DRAWINGS

FIG. 1 shows a micelle composition of the present invention, in which a drug molecule is encapsulated within the micelle, and the micelle comprises Ligand-PEG-EGCG conjugate in an outer shell and oligomeric EGCG (OEGCG) in an inner shell.

FIG. 2 shows a micelle composition of the present invention, in which a drug molecule is encapsulated within the micelle, and the micelle comprises Ligand-PEG-EGCG conjugate plus bare PEG-EGCG in an outer shell and oligomeric EGCG (OEGCG) in an inner shell.

FIG. 3 shows the chemical synthesis scheme of VS7-PEG-EGCG via conjugating the N-terminal of VS7 peptide to HOOC-PEG-EGCG.

FIG. 4 shows the chemical synthesis scheme of CST-PEG-EGCG via conjugating the N-terminal of CST peptide to HOOC-PEG-EGCG.

FIG. 5 shows the chemical synthesis scheme of CLI-PEG-EGCG via conjugating the N-terminal of CLI peptide to HOOC-PEG-EGCG.

FIG. 6 shows the successful formulation of VS7-MINC-doxorubicin (A), CST-MINC-doxorubicin (B) and CLI-MINC-doxorubicin (C).

FIG. 7 shows the cardio myoblast cell line (H9C2) uptake of CST-MINC-doxorubicin, CLI-MINC-doxorubicin, and VS7-MINC-doxorubicin by measuring fluorescence signals.

FIG. 8 shows the chemical synthesis scheme of VS7-PEG-EGCG via conjugating the C-terminal of VS7 peptide to HO-PEG-EGCG.

FIG. 9 shows the chemical synthesis scheme of CST-PEG-EGCG via conjugating the C-terminal of CST peptide to HO-PEG-EGCG.

FIG. 10 shows the chemical synthesis scheme of CLI-PEG-EGCG via conjugating the C-terminal of CLI peptide to HO-PEG-EGCG.

FIG. 11 shows the chemical synthesis scheme of VS7-PLA-EGCG via conjugating the N-terminal of VS7 peptide to HOOC-PLA-EGCG.

FIG. 12 shows the chemical synthesis scheme of VS7-PLGA-EGCG via conjugating the N-terminal of VS7 peptide to HOOC-PLGA-EGCG.

FIG. 13 shows the chemical synthesis scheme of VS7-Dextran-EGCG via conjugating the C-terminal of VS7 peptide to HO-Dextran-EGCG.

### DETAILED DESCRIPTION OF THE INVENTION

### Definitions

The term “about” is defined as ±10%, preferably ±5%, of the recited value.

The term “a CVD-targeting ligand”, as used herein, refers to a molecule of molecular weight <10000 Daltons, for example, 300-3500 Daltons, such as a peptide or a small molecule, that binds or targets receptors on a cardiovascular system, heart or endothelial cell surface.

The term “cytokines” refer to proteins (˜5-70 kDa) important in cell signaling. Cytokines have been shown to be involved in autocrine, paracrine, and endocrine signaling as immunomodulating agents.

The term “epigallocatechin gallate” refers to an ester of epigallocatechin and gallic acid, and is used interchangeably with “epigallocatechin-3-gallate” or EGCG.

The term “oligomeric EGCG” (OEGCG) refers to 2-50 or 3-20 monomers of EGCG that are covalently linked. OEGCG preferably contains 4 to 12 monomers of EGCG.

The term “nanoparticles” refers to particles with a diameter below 1 μm and between 1-999 nm.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The term “MINC” (Multi-pathway Immune-modulating Nanocomplex Combination therapy) is a platform technology. As used in this application, MINC utilizes the bioactivity of PEG-flavonoid conjugate and oligomeric EGCG (OEGCG). MINC can encapsulate additional CVD treating agents to form MINC-agent.

The term “MINC-agent”, as used in this application, is a micelle with a shell formed by CVD-targeting ligand-PEG-flavonoid conjugate and optionally oligomeric flavonoid such as OEGCG and has an agent encapsulated within the shell.

### Flavonoids

Flavonoids suitable for the present invention have the general structure of Formula I:

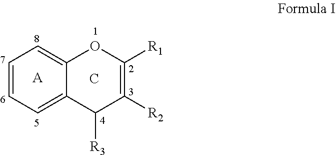

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

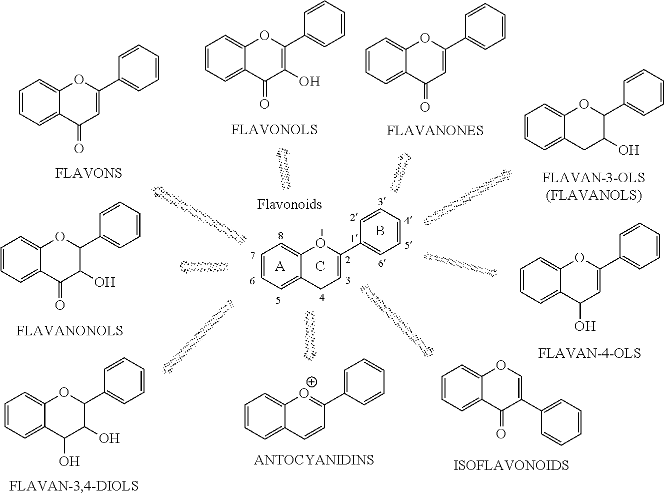

Preferred flavonoid compounds of Formula I include: EGCG (CAS #989-51-5). EC (CAS #490-46-0), EGC (CAS #970-74-1) or ECG (CAS #1257-08-5)

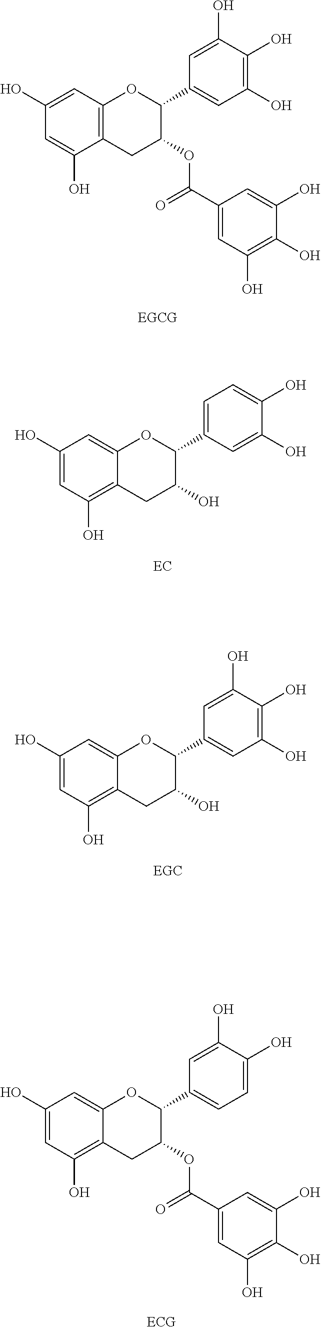 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Conjugate

The present invention provides a conjugate comprising: (a) a CVD-targeting ligand, (b) a hydrophilic polymer of polyethylene glycol (PEG), polylactic acid (PLA), polylactic-co-glycolic acid (PLGA), or dextran, and (c) a flavonoid of Formula I, wherein the PEG covalently binds to the flavonoid and the CVD-targeting ligand.

The conjugate targets CVD by the CVD-targeting ligand, and delivers active ingredients to the cardiovascular tissue to treat a CVD.

The CVD-targeting ligand is covalently linked to PEG, PLA, PLGA, or dextran, either through its-COOH groups or its —NH₂ groups by a standard chemistry known to a person skilled in the art. The molecular weight of the hydrophilic polymer in the conjugate is in general 1K-100K, preferably 3K-80K, and more preferably 5K-40K.

The flavonoid in the conjugate has a general formula (I), and is preferably EGCG, EC, EGC, or ECG. In one embodiment, the flavonoid is epigallocatechin gallate (EGCG).

In one embodiment, PEG contains an aldehyde group which is conjugated to the 5, 6, 7, or 8 position (preferably 6 or 8 position) of the A ring of the flavonoid compound.

In another embodiment, PEG contains a thiol group which is conjugated to R₁ or R₂ of the B-ring of a flavonoid (when R₁ or R₂ is —OH).

In one embodiment, the conjugate comprises PEG-EGCG, which is PEG linked to one or two molecules of EGCG; which can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R₁ or R₂ of Formula I, wherein, R₁ or R₂ is a phenyl group. See WO2015/171079.

In another embodiment, the conjugate comprises PEG-EC, PEG-EGC, or PEG-ECG, and the conjugate can be prepared by conjugating aldehyde-terminated PEG to EC, EGC, or ECG by attachment of the PEG via reaction of the free aldehyde group with the 5, 6, 7, or 8 position (preferably 6 or 8 position) of Formula I.

HOOC-PEG-CHO and HO-PEG-CHO are commonly available. In one embodiment, HOOC-PEG-CHO is conjugated to EGCG, EC, EGC, or ECG according to WO2006/124000 and WO2009/054813. HOOC-PEG-flavonoid has COOH group to react with the N terminal of a CVD-targeting peptide. In general, a CVD-targeting peptide is incubated with HOOC-PEG-flavonoid, N,N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CVD-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In another embodiment, HO-PEG-CHO is conjugated to EGCG, EC, EGC, or ECG according to WO2006/124000 and WO2009/054813. HO-PEG-flavonoid has OH group to react with the C terminal of a CVD-targeting peptide. In general, a peptide is incubated with HO-PEG-EGCG, and N,N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the CVD-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH on PEG, because the primary OH group on PEG is more reactive than the tertiary OH in the aromatic ring of flavonoid.

HOOC-PLA-CHO, HOOC-PLGA-CHO, and HO-Dextran-CHO are commercially available.

In one embodiment, HOOC-PLA-CHO is conjugated to EGCG, EC, EGC, or ECG according to WO2006/124000 and WO2009/054813. HOOC-PLA-flavonoid has COOH group to react with the N terminal of a CVD-targeting peptide. In general, a CVD-targeting peptide is incubated with HOOC-PLA-flavonoid, N,N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CVD-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HOOC-PLGA-CHO is conjugated to EGCG, EC, EGC, or ECG according to WO2006/124000 and WO2009/054813. HOOC-PLGA-flavonoid has COOH group to react with the N terminal of a CVD-targeting peptide. In general, a CVD-targeting peptide is incubated with HOOC-PLGA-flavonoid, N,N′-dicyclohexylcarbodiimide (DCC), and N-Hydroxysuccinimide (NHS) in DMSO. The reaction is stirred at room temperature, prevented from light, under nitrogen. The reaction mixture is dialyzed (membrane Mw cutoff=2000 Da) against methanol and distilled water. Next, the solution is freeze-dried to obtain lyophilized powder. To avoid self-reaction of the peptide, the C terminal of the CVD-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition.

In one embodiment, HO-Dextran-CHO is conjugated to EGCG, EC, EGC, or ECG according to WO2006/124000 and WO2009/054813. HO-Dextran-flavonoid has OH group to react with the C terminal of a CVD-targeting peptide. In general, a peptide is incubated with HO-Dextran-EGCG, and N,N′-dicyclohexylcarbodiimide (DCC) in DMSO. The reaction is stirred at room temperature, prevented from light, under nitrogen. The reaction mixture is dialyzed (membrane Mw cutoff=2000 Da) against methanol distilled water. Next, the solution is freeze-dried to obtain lyophilized powder. To avoid self-reaction of the peptide, the N terminal of the CVD-targeting peptide may be protected, for example, by resin during the reaction. Merrifield, hydroxymethyl polystyrene, PAM and MBHA resins are commonly used for preventing unwanted peptide conjugation. After the reaction, the resin can be removed under acidic condition. In this reaction, COOH group on the peptide selectively reacts with OH in the CH₂OH terminal of dextran, because this is the only primary OH group in dextran, which is more reactive than other secondary OH in dextran and tertiary OH in the aromatic ring of flavonoid.

### CVD Targeting Ligand

The CVD targeting ligand in the present invention is a ligand selected to target receptors of cardiovascular cells surface or the surrounding heart environment, including but not limited to, cardiomyocytes, vasculature cells (endothelial cells, pericytes), fibroblasts, immune cells (macrophages, T cells, B cells and mast cells), conduction system cells (AV node, sinoatrial node, purkinje fibres), and nervous system cells (glial cells, neurons) in CVD lesions.

The receptors to be targeted by CVD targeting ligand include, but not limited to, Guanylyl cyclase A (GC-A) receptors, Angiotensin II receptor (AT1R), matrix metalloproteinases (MMPs), cysteine-rich protein 2 (CRIP2), TNF receptors (e.g. TNFR1, TNFR2), BDNF receptor, Toll-like receptors (TLRs), protease-activated receptors (PARs), endothelin receptors (ETA/ETB receptors), TAM receptors, peroxisome proliferator-activated receptors (PPARs), ryanodine receptors (RyRs), thromboxane receptor, chemokine receptors (CCRs, e.g. CCR2), pattern-recognition receptors (PRRs), adrenergic receptors (e.g. β-Adrenergic receptor), cytokine receptors (CRs, e.g. IL-1R, IL-2R, IL-4R), prostaglandin receptors (e.g. prostaglandin E2 receptor), serotoninergic receptors (e.g. 5-HT2 receptor), platelet fibrinogen receptor, corticosteroid receptors (e.g. glucocorticoid receptor), nucleotide-binding oligomerization domain-like receptors (NLRs), class A1 scavenger Receptors (SR-A1), scavenger receptors, adiponectin receptor, death receptors (e.g. DR4, DR5), purinergic receptor (e.g. P2X7), G-protein-coupled receptors (GPCRs), NOTCH receptors, epidermal growth factor receptors (EGFRs), LDL receptor, platelet receptors, leptin receptors, estrogen receptor, vascular endothelial growth factor receptor (VEGFR), vascular cell adhesion protein (VCAM-1), intercellular adhesion molecule (ICAM-1), integrin (e.g. αvβ3), thrombin, stabilin-2.

In one embodiment, the heart-targeting ligand is VS7 peptide having the amino acid sequence of VVLVTSS (SEQ ID NO: 1), which targets cardiac endothelium or microenvironment in cardiac tissue.

In one embodiment, the heart-targeting ligand is CST peptide having the amino acid sequence of CSTSMLKAC (SEQ ID NO: 2), which targets alpha-B crystalline in cardiac tissue.

In one embodiment, the heart-targeting ligand is CLI peptide having the amino acid sequence of CLIDLHVMC (SEQ ID NO: 3), which targets cardiac cells or microenvironment.

In one embodiment, the heart-targeting ligand is CTT peptide having the amino acid sequence of CTTHWGFTLC (SEQ ID NO: 4), which targets MMP2 and MMP9 in cardiac tissue.

In one embodiment, the heart-targeting ligand is atrial natriuretic peptide (ANP) peptide having the amino acid sequence of SLRRSSCFGGRMDRIGAQSGLGCNSFRY (SEQ ID NO: 5), which targets guanylyl cyclase A (GC-A) receptors in cardiac tissue.

In one embodiment, the heart-targeting ligand is CRS peptide having the amino acid sequence of CRSWNKADNRSC (SEQ ID NO: 6), which targets cardiac endothelium or microenvironment in cardiac tissue.

In one embodiment, the heart-targeting ligand is DF8 peptide having the amino acid sequence of DRVYIHPF (SEQ ID NO: 7), which targets AT1R in cardiac tissue.

In one embodiment, the heart-targeting ligand is CRP peptide having the amino acid sequence of CRPPR (SEQ ID NO: 8), which targets CRIP2 in cardiac tissue.

In one embodiment, the heart-targeting ligand is QV7 peptide having the amino acid sequence of QAQGQLV (SEQ ID NO: 9), which targets TNF-α receptor in cardiac tissue.

In one embodiment, the heart-targeting ligand is AV7 peptide having the amino acid sequence of ARRGQAV (SEQ ID NO: 10), which targets BDNF receptor in cardiac tissue.

In one embodiment, the heart-targeting ligand is GV7 peptide having the amino acid sequence of GRRFIRV (SEQ ID NO: 11), which targets BDNF receptor in cardiac tissue.

In one embodiment, the heart-targeting ligand is VR7 peptide having the amino acid sequence of VHPKQHR (SEQ ID NO: 12), which targets VCAM-1 in cardiac tissue.

In one embodiment, the heart-targeting ligand is VK7 peptide having the amino acid sequence of VHSPNKK (SEQ ID NO: 13), which targets VCAM-1 in cardiac tissue.

In one embodiment, the heart-targeting ligand is CC9 peptide having the amino acid sequence of CNNSKSHTC (SEQ ID NO: 14), which targets VCAM-1 in cardiac tissue.

In one embodiment, the heart-targeting ligand is NA17 peptide having the amino acid sequence of NNQKIVNLKEKVAQLEA (SEQ ID NO: 15), which targets ICAM-1 in cardiac tissue.

In one embodiment, the heart-targeting ligand is RGD peptide having the amino acid sequence of RGD, which targets integrin in cardiac tissue.

In one embodiment, the heart-targeting ligand is GC11 peptide having the amino acid sequence of GPXRSGGGGKC (SEQ ID NO: 16), which targets thrombin in cardiac tissue.

In one embodiment, the heart-targeting ligand is KL9 peptide having the amino acid sequence of KKLVPRGSL (SEQ ID NO: 17), which targets thrombin in cardiac tissue.

In one embodiment, the heart-targeting ligand is CRK peptide having the amino acid sequence of CRKRLDRNC (SEQ ID NO: 18), which targets IL-4 receptor in cardiac tissue.

In one embodiment, the heart-targeting ligand is CRT peptide having the amino acid sequence of CRTLTVRKC (SEQ ID NO: 19), which targets Stabilin-2 in cardiac tissue.

In one embodiment, the heart-targeting ligand is CKR peptide having the amino acid sequence of CKRAVR (SEQ ID NO: 20), which targets cardiac endothelium or microenvironment in cardiac tissue.

In one embodiment, the heart-targeting ligand is CPK peptide having the amino acid sequence of CPKTRRVPC (SEQ ID NO: 21), which targets cardiac endothelium or microenvironment in cardiac tissue.

In one embodiment, the heart-targeting ligand is CAR peptide having the amino acid sequence of CARPAR (SEQ ID NO: 22), which targets cardiac endothelium or microenvironment in cardiac tissue.

In one embodiment, the heart-targeting ligand is CRS9 peptide having the amino acid sequence of CRSTRANPC (SEQ ID NO: 23), which targets cardiac endothelium or microenvironment in cardiac tissue.

In one embodiment, the heart-targeting ligand is GQ7 peptide having the amino acid sequence of GGGVFWQ (SEQ ID NO: 24), which targets cardiac endothelium or microenvironment in cardiac tissue.

In one embodiment, the heart-targeting ligand is HH7 peptide having the amino acid sequence of HGRVRPH (SEQ ID NO: 25), which targets cardiac endothelium or microenvironment in cardiac tissue.

In one embodiment, the heart-targeting ligand is CLH peptide having the amino acid sequence of CLHRGNSC (SEQ ID NO: 26), which targets cardiac endothelium or microenvironment in cardiac tissue.

In one embodiment, the heart-targeting ligand is CRS12 peptide having the amino acid sequence of CRSWNKADNRSC (SEQ ID NO: 27), which targets cardiac endothelium or microenvironment in cardiac tissue.

### Nanoparticle Composition

The term “MINC” (Multi-pathway Immune-modulating Nanocomplex Combination therapy) is a platform technology. The present invention provides a nanoparticle micelle (MINC) composition. The micelle comprises CVD-targeting ligand-PEG-flavonoid conjugate in an outer shell and oligomeric EGCG (OEGCG) in an inner shell (see FIG. 1). The CVD-targeting ligand allows the nanoparticle composition to specifically target the cardiovascular tissues.

In one embodiment, the micelle composition comprises both the outer shell and the inner shell as described above; the composition optionally has a drug encapsulated with the shells.

In one embodiment, the micelle composition comprises the outer shell as described above and does not have an inner shell; the composition optionally has a drug encapsulated with the shell.

In one embodiment, the micelle composition comprises CVD-targeting ligand-polymer-flavonoid conjugate in an outer shell and oligomeric flavonoid in an inner shell, wherein the flavonoid in the outer shell and the flavonoid in the inner shell are independently EGCG, EC, EGC, or ECG, and the polymer is PEG, PLA, PLGA, or dextran. A preferred polymer is PEG. A preferred flavonoid is EGCG. FIG. 1 shows a preferred micelle composition. The CVD-targeting ligand allows the nanoparticle composition to specifically target the CVD tissues.

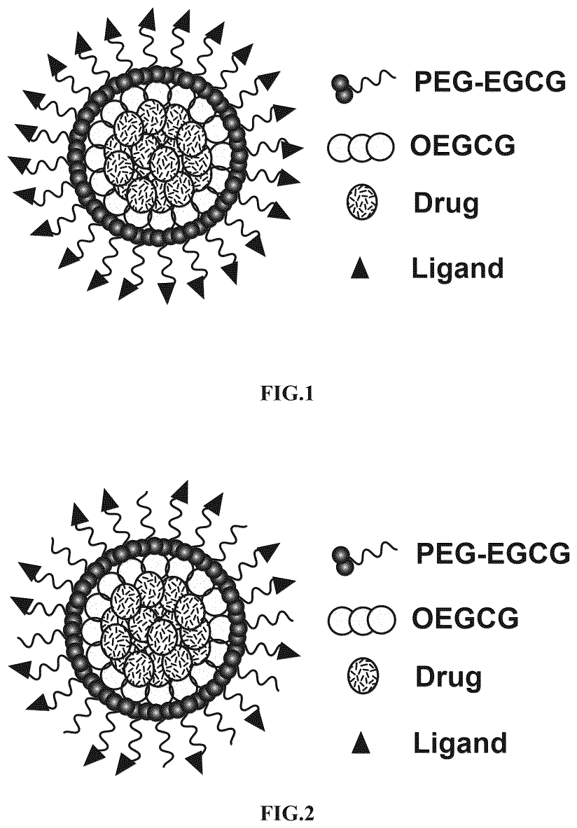

In one embodiment, the micelle outer shell further comprises a bare PEG-flavonoid conjugate such as PEG-EGCG, which does not have a CVD-targeting ligand linked to PEG-flavonoid. See FIG. 2. In such a micelle outer shell, the ratio of ligand-PEG-EGCG to ligand-PEG-EGCG plus PEG-EGCG is in general more than 10%, or more than 20%, or more than 30%, or more than 50%, and up to 100%. In one embodiment, the ratio of ligand-PEG-EGCG to ligand-PEG-EGCG plus PEG-EGCG is 10-90%, or 20-80%, or 40-60%.

In one embodiment, the micelle shell comprises two or more different ligand-polymer-flavonoid conjugates, in that the CVD-targeting ligands are different and they target to different receptors of cardiovascular cells.

The micelles optionally comprise a CVD-treating molecule (an agent) encapsulated within the micelle (MINC-agent)

In one embodiment, the MINC-agent composition comprises three active ingredients, which are complementary in function to tackle both immune response and signaling pathways by its backbone components (PEG-flavonoid/OEGCG), and additional signaling pathways by a selected drug molecule for treating CVD. Each nanoparticle is a fixed-dose combination drug with the three active ingredients at fixed molar ratio.

The present invention delivers MINC-agent to targeted cardiovascular tissues by active delivery of the micelles through a CVD-targeting ligand to cardiovascular system with specific receptors.

In one embodiment, the nanocomplex shell of the present invention contains the first two active ingredients, OEGCG and PEG-flavonoid (e.g., PEG-EGCG) in the backbone of the micelle composition. They are derivatives of EGCG, which is a strong immune modulator and regulates a wide spectrum of disease signaling pathways. EGCG regulates both innate and adaptive immunity. However, the bioavailability of EGCG is low and EGCG is not stable. The present nanocomplex composition overcomes the bioavailability issue of EGCG by forming a nanocarrier to carry EGCG to a target site for treatment, and overcomes the stability issue of EGCG by forming OEGCG and PEG-EGCG complex, which effectively enables EGCG as highly effective therapeutic agents.

The nanocomplex of the present invention optionally contains a third active ingredient, which is a drug molecule encapsulated in the nanoparticles for treating CVD.

The agent, as used herein, refers to a molecule that have a therapeutic activity (e.g., a drug for treating a CVD. For example, the encapsulated agents can be various chemicals including small molecule chemicals, peptides, proteins, monoclonal antibodies, RNA/DNA, and vaccine.

The therapeutic agents effective in inhibiting the progress of CVD and promoting cardiovascular regeneration, include but not limited, to IGF-1, TGF-β1, MCP-1, TIMP-1, VEGF, β-FGF, Endothelin-1, urocortin, VEGF, HGF, FGF, PDGF, TGF-β, neuregulin, NO-synthase, Evasin-3, Evasin-4, IL-1 trap, IL-1RA, anti-IL-1β, anti-IL-8, anti-TNF-α, anti-CCL-5, anti-MCP-1, anti-CXCR2, anti-IL-6R, IL-19, anti-IL-12, anti-IL-23, anti-MMP9, anti-IL-1β, anti-TNF-α, anti-IL-1β, anti-IL-6, anti-IL-7, anti-IL-12 or anti-IL-23, sacubitril, valsartan, aliskiren, enoxaparin, clopidogrel, abciximab, eptifibatide, bivalirudin, morphine, cyclosporine A, furosemide, staphylokinase, dapagliflozin, anakinra, canakinumab, rimonabant, losmapimod, darapladib, SERCA2a gene or IONIS-AGT-LRx siRNA.

In one embodiment, the MINC-agent comprises the CVD-targeting ligand of CTT, CST, CRS, DF8, CRP, CKR, CPK, CAR, CRS9, CLI, GQ7, HH7, VS7, CLH, CRS12, QV7, AV7 or GV7 and the agent of anti-CD3, anti-CD3, anti-CD39, anti-CD73, anti-PD-1, anti-PD-L1, anti-PD-L2, anti-CTLA4, anti-GZM A, anti-GZM B, IGF-1 or anti-TNF-α; such agent is suitable for promoting cardiomyocyte survival for treating myocardial infarction, heart failure and other cardiac-related diseases.

In one embodiment, the MINC-agent comprises the CVD-targeting ligand of CTT, CST, CRS, DF8, CRP, CKR, CPK, CAR, CRS9, CLI, GQ7, HH7, VS7, CLH, CRS12, QV7, AV7 or GV7 and the agent of SERCA2a gene or IONIS-AGT-LRx antisense RNA for treating heart failure.

In one embodiment, the MINC-agent comprises the CVD-targeting ligand of CTT, CST, CRS, DF8, CRP, CKR, CPK, CAR, CRS9, CLI, GQ7, HH7, VS7, CLH, CRS12, QV7, AV7 or GV7 and the agent of Evasin-3, Evasin-4, IL-1 trap, IL-1RA, anti-CD3, anti-CD3, anti-CD39, anti-CD73, anti-PD-1, anti-PD-L1, anti-PD-L2, anti-CTLA4, anti-GZM A, anti-GZM B, anti-IL-1β, TGF-β1, MCP-1, anti-IL-8, anti-IL-6, anti-IL-10, anti-MCP-1, anti-CXCR2, TIMP-1, VEGF, β-FGF or anti-TNF-α; such MINC-Agent is suitable for reducing inflammation for treating myocardial infarction, heart failure and other cardiac-related diseases.

In one embodiment, the MINC-agent comprises the CVD-targeting ligand of CTT, CST, CRS, DF8, CRP, CKR, CPK, CAR, CRS9, CLI, GQ7, HH7, VS7, CLH, CRS12, QV7, AV7 or GV7 and the agent of endothelin-1, urocortin, VEGF, HGF, FGF, PDGF, TGF-β, neuregulin, NO-synthase, FGF or IGF-1; such MINC-Agent is suitable for promoting cardiac cell growth and vascularization for treating myocardial infarction, heart failure and other cardiac-related diseases.

In one embodiment, the MINC-agent comprises the CVD-targeting ligand of CTT, CST, CRS, DF8, CRP, CKR, CPK, CAR, CRS9, CLI, GQ7, HH7, VS7, CLH, CRS12, QV7, AV7 or GV7 and the agent of sacubitril, valsartan, aliskiren, enoxaparin, clopidogrel, abciximab, eptifibatide, bivalirudin, morphine, cyclosporine A, furosemide, staphylokinase, dapagliflozin, anakinra, canakinumab, rimonabant, losmapimod or darapladib; such MINC-Agent is suitable for treating myocardial infarction, heart failure and other cardiac-related diseases.

In one embodiment, the MINC-agent comprises VR7, VK7, CC9, RGD, GC11, KL9, CRK or CRT and the agent of anti-CD3, anti-CD3, anti-CD39, anti-CD73, anti-PD-1, anti-PD-L1, anti-PD-L2, anti-CTLA4, anti-GZM A, anti-GZM B, anti-PCSK9, anti-endothelial lipase, anti-IL-1, anti-IL-4, anti-IL5, anti-IL-6, anti-IL-10, anti IL-13, anti-IIL-17, anti-IL-19, anti-TNF-α, anti-TNF-β, anti-TNF-γ, anti IFN-γ or anti-TGFB; such MINC-Agent is suitable for treating atherosclerosis and prevent the progression of CVD.

In one embodiment, the MINC-agent comprises RGD and the agent of anti-elastin, anti-TNF-α, anti-IL-8, anti-IL-1β, anti-IL-6, anti-IL-17 or anti-MCP-1; such MINC-Agent is suitable for treating aneurysm.

The nanoparticle composition of the present invention is stable in a hydrophilic environment, such as blood circulation, and dissociates in a hydrophobic environment, such as a heart tissue. Once the nanoparticle complex enters tissue which is hydrophobic, it dissociates and frees its active components OEGCG, PEG-flavonoid such as PEG-EGCG, and the drug molecule in the nanocomplex. The free active components regain their bioactivities in CVD retardation.

### Process for Preparing the Nanoparticle Composition

The nanoparticle composition of the present invention can be prepared by a process comprises the steps of: (a) mixing a drug molecule with flavonoid oligomer (e.g., OEGCG) and the CVD-targeting ligand conjugate of the present invention in an aqueous solution; and (b) filtering the mixture through a membrane with a molecular weight cut-off of 8,000-300,000 daltons to remove small molecular weight molecules and retain large molecular weight molecules.

In one preferred embodiment, the process further comprises step (c), filtering the large molecular weight molecules through 0.2-0.3 μm membrane and collecting the filtrate.

The present process optionally further comprises a lyophilization step (d) after step (c). Step (d): lyophilizing the filtrate by stepwise freezing at (i) about 0-5° C., (ii) about −20 to −30° C., and (iii) at about −60 to −100° C., and then drying.

In step (a), the drug molecule is dissolved in an aqueous solvent, such as phosphate-buffer saline, saline, water, bicarbonate buffer, oxyhemoglobin buffer, bis-tris alkane, Tris-HCl, HEPES, histidine buffer, NP-40, RIPA (radioimmunoprecipitation assay buffer), tricine, TES, TAPS, TAPSO, Bicine, MOPS, PIPES, cacodylate, or MES. Preferred solvents are phosphate-buffer saline, saline, or water. The protein drug concentration is in general 0.01-50 mg/ml, preferred 0.05-10 mg/ml, and more preferred 0.1-5 mg/ml.

The flavonoid oligomer and the CVD-targeting ligand conjugate are dissolved in ketones, acetonitrile, alcohols, aldehydes, ethers, acetates, sulfoxides, benzenes, organic acids, amides, aqueous buffers, and any combination thereof. Preferred solvents are alcohols, acetonitrile, sulfoxides, amides, and any combination thereof. The OEGCG/EGCG and PEG-EGCG concentrations are in general independently 0.001-10 mg/ml, preferred 0.005-1 mg/ml, or 0.1-5 mg/ml.

It is important that OEGCG is in molar excess of the drug agent. In general, the molar ratio of the EGCG in OEGCG to the drug molecule is between 1-500 to 1, 2-500 to 1, 3-500 to 1, or 5-500 to 1, preferably 3-100 to 1, 5-100 to 1, or 10-50 to 1. The molar ratio is calculated by the number of moles of monomer EGCG in OEGCG to the number of moles of the drug molecule. The molar excess of EGCG ensures most or all drug agents are encapsulated by the OEGCG molecules. Unencapsulated drug agents, which would not be selectively distributed to target tissue and would cause lower efficacy and safety issues, are avoided by controlling the molar ratio of OEGCG to protein in the present process.

In step (b), the above mixture is filtered through a membrane with a molecular weight cut-off between 8,000-300,000 daltons, preferably between 8,000-200,000 daltons, 8,000-150,000 daltons, or 8,000-12,000 daltons, to remove small molecular weight molecules and retain large molecular weight molecules. The ultrafiltration membrane material is selected from the group consisting of cellulose (and its derivatives), polyethersulfone (PES), polytetrafluoroethylene (PTFE), nylon, polyvinylidene fluoride or polyvinylidene difluoride (PVDF), and polypropylene (PP); preferably cellulose (and its derivatives), PTFE, and PVDF.

The mixture is optionally diluted in an aqueous solvent such as those described above in step (a) before ultrafiltration.

The ultrafiltration step (b) removes unwanted impurities of small molecular weight, such as unreacted OEGCG or EGCG, or reaction by-products. These impurities may reduce drug efficacy and safety. The excess of unreacted OEGCG or EGCG may also lead to aggregation of the individual nanoparticles to about 1000 nm size particles, which would reduce efficacy and cause potential toxicity.

In step (c), the retained large molecular weight molecules are filtered through a membrane having a pore size of about 0.2-0.3 μm, such as 0.22 μm, and the filtrate is collected. This is to remove unwanted impurities of large molecular sizes, such as mega-aggregates. These aggregates may be excreted from entering tissues due to its mega size. These aggregates reduce overall efficacy/safety and have a higher chance of inducing immunogenicity to the patients. Large size nanoparticles are also easier to be taken up by RES in the liver, lungs, and more undesired organs.

The membrane material of step (c) is selected from the group consisting of cellulose (and its derivatives), PES, PTFE, nylon, PVDF, and PP; preferably cellulose (and its derivatives), PES, and PP.

In one embodiment, the steps (b) and (c) are repeated at least one time, for example, repeated 1, 2, 3, or 4 times before step (d), to effectively remove unwanted small molecule impurities and large aggregates.

After step (c), the filtrate is stored at 2-8° C.

The present process optionally further comprises a lyophilization step (d) after step (c) to provide a long-term stability of the nanoparticle composition.

### Pharmaceutical Compositions

The present invention provides pharmaceutical compositions comprising the nanoparticle composition of the present invention and optionally one or more pharmaceutically acceptable carriers. The nanoparticle composition in a pharmaceutical composition in general is about 1-90%, preferably 20-90%, or 30-80% for a tablet, powder, or parenteral formulation. The nanoparticle composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

### Method of Treatment

The present invention is directed to a method of treating CVD diseases, comprising the step of administering an effective amount of the nanoparticle composition of the present invention to a subject in need thereof. Suitable CVD to be treated by the present invention include but not limited to coronary artery disease, myocardial infarction, heart failure, aortic aneurysm and atherosclerosis.

“An effective amount,” as used in this application, is the amount effective to treat a disease by ameliorating the pathological condition or reducing the symptoms of the disease.

Dosing for a ligand-polymer-flavonoid, e.g., ligand-polymer-EGGC, for injection, is in general 0.01 to 100 mg/kg (total weight of the polymer-flavonoid/subject body weight), or 0.01 to 1000 mg/kg.

In one embodiment, the method comprises the step of administering to a subject in need thereof an effective amount of MINC having a shell formed by one or more ligand-hydrophilic polymer-flavonoid conjugates and optionally with a bare polymer-flavonoid conjugate, with or without flavonoid oligomers, optionally having an agent encapsulated within the shell.

In one embodiment, the shell is formed by ligand-PEG-EGCG, optionally with PEG-EGCG.

In one embodiment, the shell is formed by ligand-PEG-EGCG and OEGCG, and optionally PEG-EGCG.

Dosing of the MINC-agent is based on the known dosage of the agent for treating a particular disease and the subject condition. The dosage can be a food drug administration (FDA) approved dosage or a dosage used in clinical trial.

In MINC-agent, the total weight of ligand-PEG-EGCG and PEG-EGCG if present, is close to the encapsulated drug agent. The weight of OEGCG, if present, varies. In general, the dosage of ligand-PEG-EGCG and PEG-EGCG if present, combined with OEGCG is between 0.01 to 1000 mg/kg.

The concentration for the encapsulated drug agents can be as low as 0.01 mg/kg (e.g., for cytokine drugs) and as high as 100 mg/kg (for antibody drugs).

For treating myocardial infarction, anti-IL-6 is given at 0.01-100 mg/kg or 0.01-1000 mg/kg IV one to three times per week. The effective dose of AT-MINC-IL-6 in the same dose range can be used for treating myocardial infarction.

For treating heart failure or coronary artery disease, anti-IL-1ß is given at 0.01-100 mg/kg or 0.01-1000 mg/kg IV one to three times per week. The effective dose of AT-MINC-anti-IL-1 in the same dose range can be used for treating heart failure, coronary artery diseases or aortic aneurysm.

For treating atherosclerosis, anti-EL (anti-endothelial lipase) is given at 0.01-100 mg/kg or 0.01-1000 mg/kg IV every one to three months. The effective dose of MINC-anti-EL in the same dose range can be used for treating atherosclerosis.

The present invention is useful in treating human and non-human animals. For example, the present invention is useful in treating a mammal subject, such as humans, horses, pigs, cats, and dogs.

The following examples further illustrate the present invention. These examples are intended merely to be illustrative of the present invention and are not to be construed as being limiting.

### EXAMPLES

### Example 1: VS7 Peptide Conjugation to HOOC-PEG-EGCG

### Materials

VS7 peptide was purchased from Hangzhou Xinbosi Biomedical

HOOC-PEG-CHO was purchased from NBC chemical

### Method

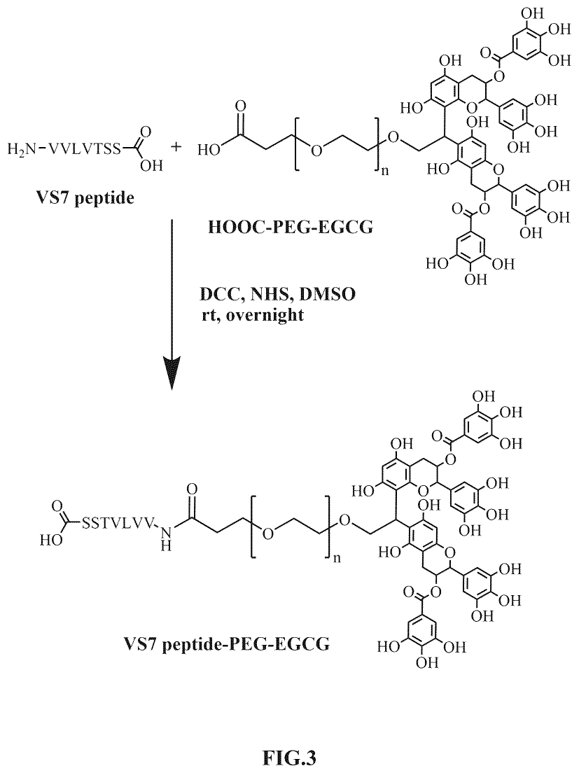

HOOC-PEG-CHO was conjugated to EGCG according to WO2006/124000 and WO2009/054813; VS7 peptide was conjugated to HOOC-PEG-EGCG via the conjugation between COOH group on PEG and NH₂ group on VS7 to form VS7-PEG-EGCG (N′ linked) (FIG. 3).

Specifically, 1-1000 mg VS7 was PEGylated by incubation with 1-1000 mg HOOC-PEG-EGCG, 1-1000 mg N,N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of VS7-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after VS7 conjugation, a new peak with retention time at 7.08 min was present. The HPLC results indicate a successful conjugation of VS7-PEG-EGCG.

### Example 2: CST Peptide Conjugation to HOOC-PEG-EGCG

### Materials

CST peptide was purchased from Hangzhou Xinbosi Biomedical

HOOC-PEG-CHO was purchased from NBC chemical

### Method

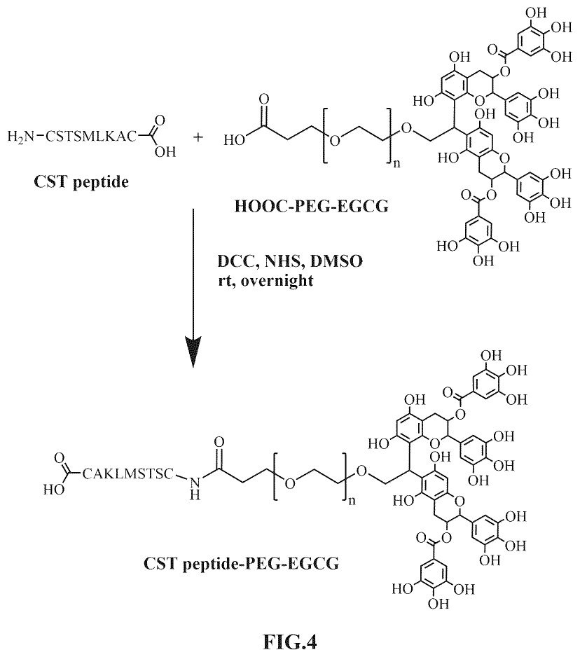

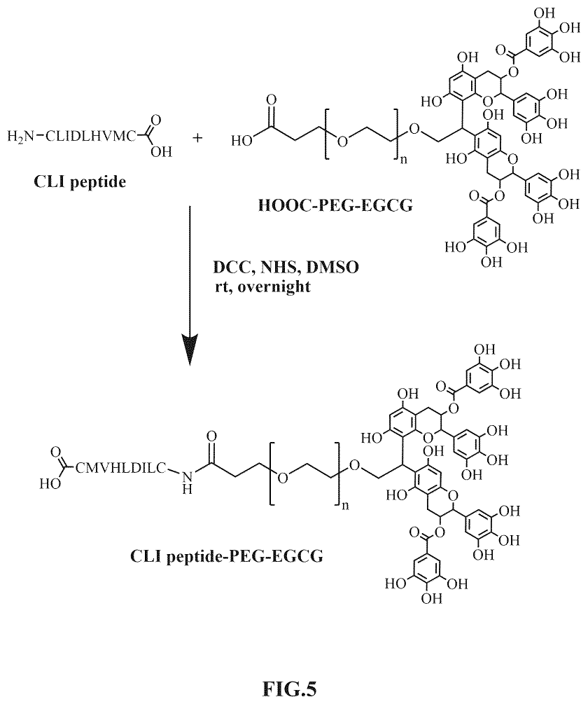

HOOC-PEG-CHO was conjugated to EGCG according to WO2006/124000 and WO2009/054813; CST peptide was conjugated to HOOC-PEG-EGCG via the conjugation between COOH group on PEG and NH₂ group on CST to form CST-PEG-EGCG (N′ linked) (FIG. 4).

Specifically, 1-1000 mg CST was PEGylated by incubation with 1-1000 mg HOOC-PEG-EGCG, 1-1000 mg N,N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of CST-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after CST conjugation, a new peak with retention time at 7.06 min was present. The HPLC results indicate a successful conjugation of CST-PEG-EGCG.

### Example 3: CLI Peptide Conjugation to HOOC-PEG-EGCG

### Materials

CLI peptide was purchased from Hangzhou Xinbosi Biomedical

HOOC-PEG-CHO was purchased from NBC chemical

### Method

HOOC-PEG-CHO was conjugated to EGCG according to WO2006/124000 and WO2009/054813; CLI peptide was conjugated to HOOC-PEG-EGCG via the conjugation between COOH group on PEG and NH₂ group on CLI to form CLI-PEG-EGCG (N′ linked) (FIG. 5).

Specifically, 1-1000 mg CLI was PEGylated by incubation with 1-1000 mg HOOC-PEG-EGCG, 1-1000 mg N,N′-dicyclohexylcarbodiimide (DCC), and 1-1000 mg N-Hydroxysuccinimide (NHS) in DMSO. The reaction was stirred at room temperature, prevented from light, under nitrogen for 24 hours. The reaction mixture was dialyzed (membrane molecular weight cutoff=2000 Da) against methanol and distilled water for 3 days. Next, the solution was freeze-dried to obtain lyophilized powder.

### Results

HPLC was used to confirm the formulation of CLI-PEG-EGCG. HPLC was conducted under the following conditions: Column: C18, 4.6×150 mm, 4 μm; Elution: A=0.1% TFA in H₂O, B=0.1% TFA in ACN; Oven temperature: 40° C.; Flow speed: 1 ml/min; Autosampler temperature: 15° C.; Measurement: UV280.

HOOC-PEG-EGCG had a retention time at 6.04 min, after CLI conjugation, a new peak with retention time at 7.20 min was present. The HPLC results indicate a successful conjugation of CLI-PEG-EGCG.

### Example 4: Preparing VS7-MINC-Doxorubicin, CST-MINC-Doxorubicin and CLI-MINC-Doxorubicin

### Materials

VS7-PEG-EGCG was prepared according to Example 1.

CST-PEG-EGCG was prepared according to Example 2.

CLI-PEG-EGCG was prepared according to Example 3.

Doxorubicin was purchased from Sigma-Aldrich.

### Method

VS7-MINC-doxorubicin nanoparticles, CST-MINC-doxorubicin nanoparticles, CLI-MINC-doxorubicin nanoparticles were prepared according to the following protocol:
1. Incubate 5-500 μg doxorubicin in 1 mL DMSO for 15 min to 1 hour.
2. Add 1-100 μg of OEGCG and 1-10,000 μg of VS7-PEG-EGCG, CST-PEG-EGCG or CLI-PEG-EGCG. Incubate the mixture at 25° C. for 3 hours.
3. Filter out the liquid with a 10K MWCO filter unit. Wash the filter 3 time with 0.9% NaCl
4. Lyophilize to dry powder.

### Results

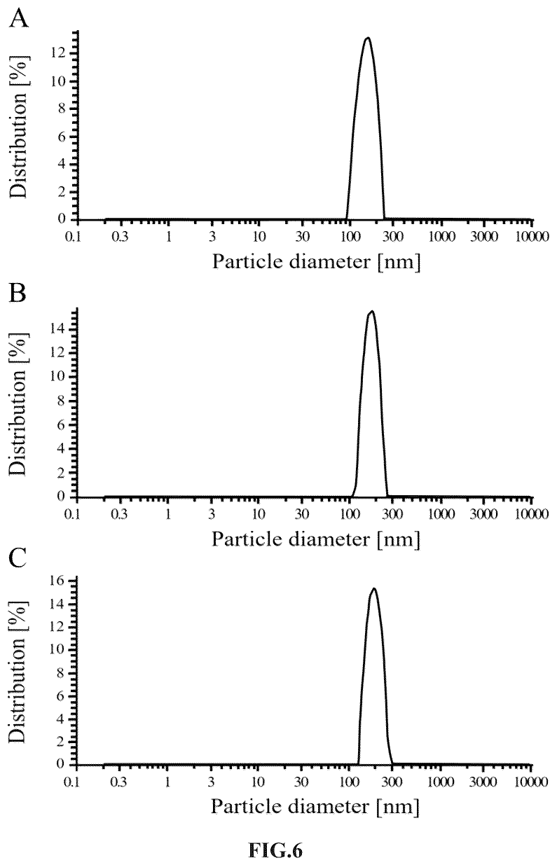

Nanoparticle size was measured by DLS (Anton Paar, Litesizer 500). FIG. 6 showed successful formulation of (A) VS7-MINC-doxorubicin, (B) CST-MINC-doxorubicin, and (C) CLI-MINC-doxorubicin.

### Example 5: Different Ligand Conjugated-MINC-Doxorubicin in Delivering Drugs into Heart Myoblast Cells

### Materials

VS7-MINC-doxorubicin, CST-MINC-doxorubicin and CLI-MINC-doxorubicin were formulated according to Example 4.

### Method

Heart myoblast cell line H9C2 was seeded at 8×103 cells/well in 96 well plate with and incubated overnight. On the second day, cells were treated with MINC-doxorubicin, VS7-MINC-doxorubicin, CST-MINC-doxorubicin or CLI-MINC-doxorubicin at 2.5 μM for 2 hours (n=2). After the treatment, the fluorescence intensity was measured by Molecular Devices Gemini XPS fluorescent microplate reader to assess the delivery efficiency. Data was shown as means±SD, and statistically analyzed by GraphPad Prism 7. The statistical significance was calculated by one-way ANOVA and differences were considered to be significant at *: p<0.05, **: p<0.01; ***: p<0.001; ****: p<0.0001.

### Result

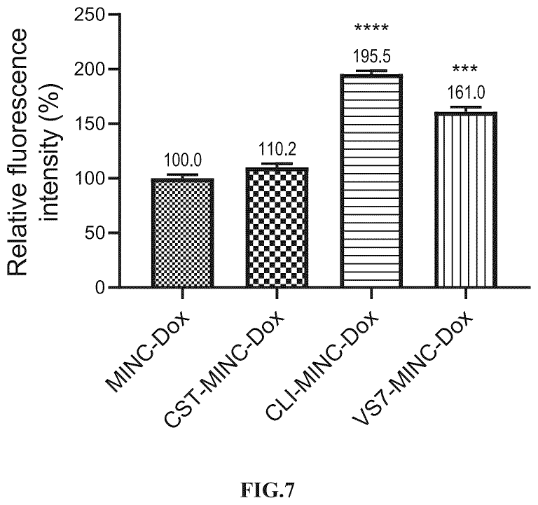

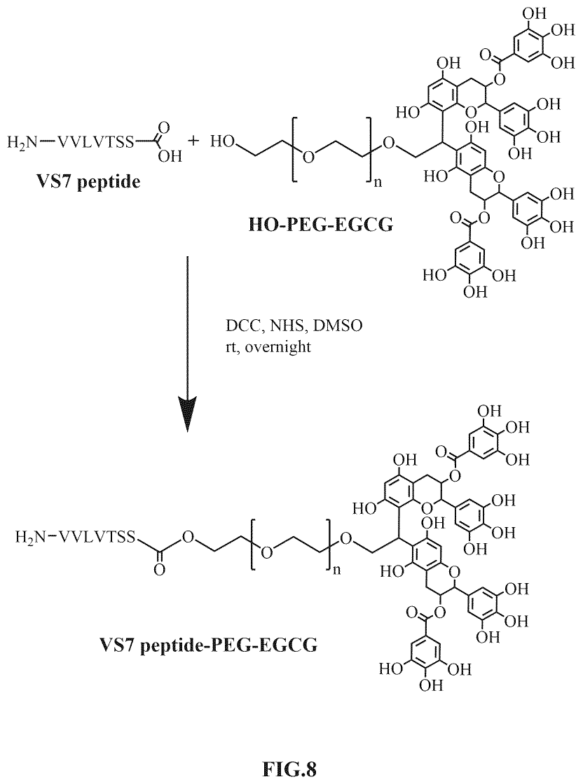

Doxorubicin is a red fluorescent compound, its delivery into cells was observed using fluorescent microplate reader. The higher fluorescence intensity means more doxorubicin was delivered into the cells. In FIG. 7, compared to MINC-doxorubicin, the fluorescent signal of VS7-MINC-doxorubicin and CLI-MINC-doxorubicin were significantly stronger in the H9C2 cells. We also observed a trend of higher fluorescence signal in CST-MINC-doxorubicin treatment cells. These results demonstrate that the heart-targeting peptides increased specific drug delivery into heart myoblast cells.

### Example 6: VS7 Peptide Conjugation to HO-PEG-EGCG (Prophetic Example)

### Objectives

This experiment is intended to demonstrate the conjugation of VS7 peptide to HO-PEG-EGCG. HPLC is used to detect the formation of new product (VS7-PEG-EGCG) with different retention time from HO-PEG-EGCG. NMR can be used to confirm the structure.

### Materials

VS7 peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

VS7 peptide is conjugated to HO-PEG-EGCG via the conjugation between OH group on PEG and COOH group on VS7 to form VS7-PEG-EGCG (C′ linked). See FIG. 8.

Specifically, 1-1000 mg VS7 is PEGylated by incubation with 1-1000 mg HO-PEG-EGCG, and 1-1000 mg N,N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 7: CST Peptide Conjugation to HO-PEG-EGCG (Prophetic Example)

### Objectives

This experiment is intended to demonstrate the conjugation of CST peptide to HO-PEG-EGCG. HPLC is used to detect the formation of new product (CST-PEG-EGCG) with different retention time from HO-PEG-EGCG. NMR can be used to confirm the structure.

### Materials

CST peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

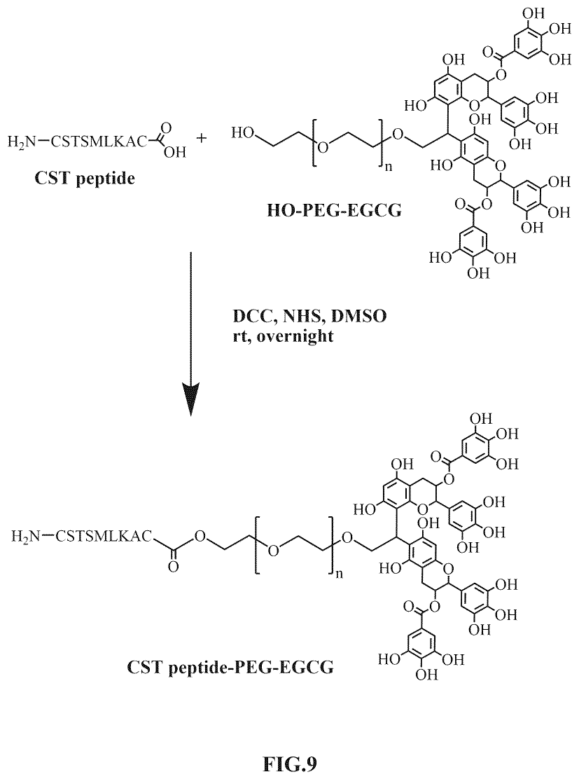

CST peptide is conjugated to HO-PEG-EGCG via the conjugation between OH group on PEG and COOH group on CST to form CST-PEG-EGCG (C′ linked). See FIG. 9.

Specifically, 1-1000 mg CST is PEGylated by incubation with 1-1000 mg HO-PEG-EGCG, and 1-1000 mg N,N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 8: CLI Peptide Conjugation to HO-PEG-EGCG (Prophetic Example)

### Objectives

This experiment is intended to demonstrate the conjugation of CLI peptide to HO-PEG-EGCG. HPLC is used to detect the formation of new product (CLI-PEG-EGCG) with different retention time from HO-PEG-EGCG. NMR can be used to confirm the structure.

### Materials

CLI peptide is purchased from Hangzhou Xinbosi Biomedical

HO-PEG-CHO is purchased from Huanteng pharma

### Method

HO-PEG-CHO is conjugated to EGCG according to WO2006/124000 and WO2009/054813 to prepare HO-PEG-EGCG.

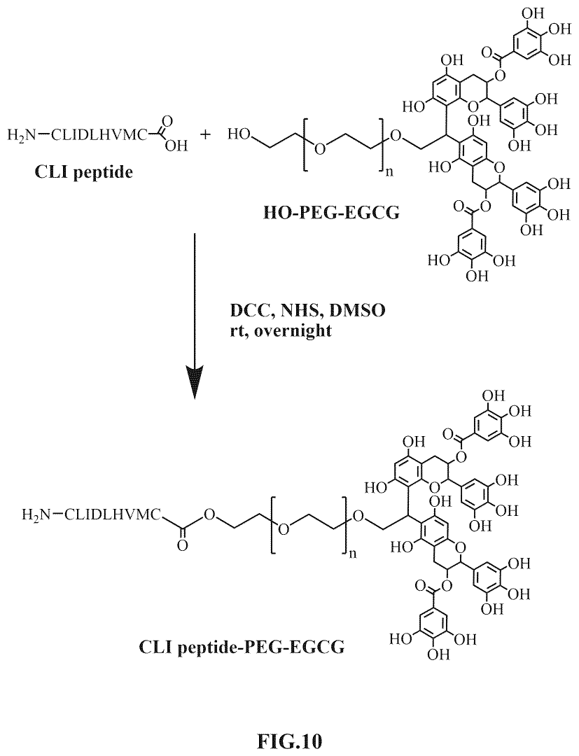

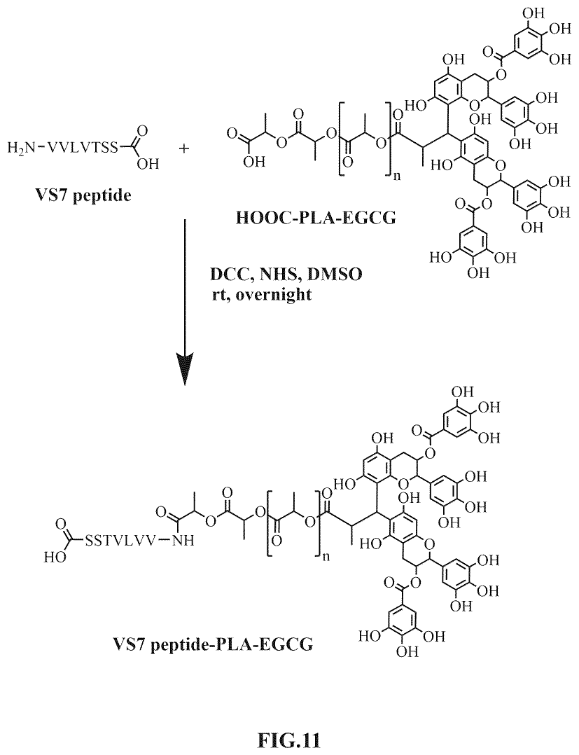

CLI peptide is conjugated to HO-PEG-EGCG via the conjugation between OH group on PEG and COOH group on CLI to form CLI-PEG-EGCG (C′ linked). See FIG. 10.

Specifically, 1-1000 mg CLI is PEGylated by incubation with 1-1000 mg HO-PEG-EGCG, and 1-1000 mg N,N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 9: VS7 Peptide Conjugation to HOOC-PLA-EGCG (Prophetic Example)

### Objectives

This experiment is intended to demonstrate conjugation of VS7 peptide to HOOC-PLA-EGCG. HPLC is used to detect the formation of new product (VS7-PLA-EGCG) with different retention time from HOOC-PLA-EGCG. NMR can be used to confirm the structure.

### Materials

VS7 peptide is purchased from Hangzhou Xinbosi Biomedical.

HOOC-PLA-CHO is purchased from Merck (Sigma-Aldrich).

### Method

HOOC-PLA-CHO is conjugated to EGCG according to WO2006/124000 and WO2009/054813; VS7 peptide is conjugated to HOOC-PLA-EGCG via the conjugation between COOH group on PLA and NH₂ group on VS7 to form VS7-PLA-EGCG (N′ linked). See FIG. 11.

Specifically, 1-1000 mg VS7 is incubated with 1-1000 mg HOOC-PLA-EGCG, 1-1000 mg N,N′-dicyclohexylcarbodiimide (DCC), and 1-1000 mg N-Hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 10: VS7 Peptide Conjugation to HOOC-PLGA-EGCG (Prophetic Example)

### Objectives

This experiment is intended to demonstrate conjugation of VS7 peptide to HOOC-PLGA-EGCG. HPLC is used to detect the formation of new product (VS7-PLGA-EGCG) with different retention time from HOOC-PLGA-EGCG. NMR can be used to confirm the structure.

### Materials

VS7 peptide is purchased from Hangzhou Xinbosi Biomedical.

HOOC-PLGA-CHO is purchased from Merck (Sigma-Aldrich).

### Method

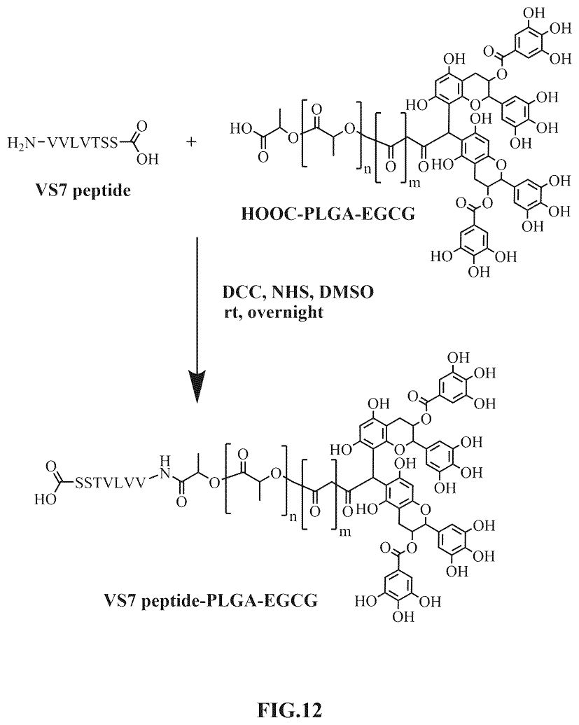

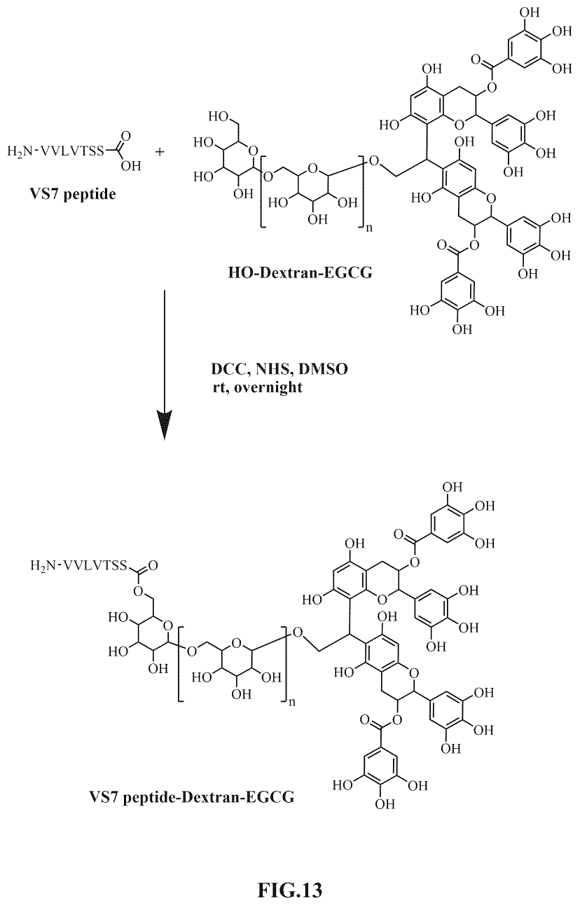

HOOC-PLGA-CHO is conjugated to EGCG according to WO2006/124000 and WO2009/054813; VS7 peptide is conjugated to HOOC-PLGA-EGCG via the conjugation between COOH group on PLGA and NH₂ group on VS7 to form VS7-PLGA-EGCG (N′ linked). See FIG. 12.

Specifically, 1-1000 mg VS7 is incubated with 1-1000 mg HOOC-PLGA-EGCG, 1-1000 mg N,N′-dicyclohexylcarbodiimide (DCC), and 1-1000 mg N-Hydroxysuccinimide (NHS) in DMSO. The reaction is stirred at room temperature, prevented from light, under nitrogen for 24 hours. The reaction mixture is dialyzed (membrane molecular weight cutoff=2000 Da) against methanol and distilled water for 3 days. Next, the solution is freeze-dried to obtain lyophilized powder.

### Example 11: VS7 Peptide Conjugation to HO-Dextran-EGCG (Prophetic Example)

### Objectives

This experiment is intended to demonstrate the conjugation of VS7 to HO-Dextran-EGCG. HPLC is used to detect the formation of new product (VS7-Dextran-EGCG) with different retention time from HO-Dextran-EGCG. NMR can be used to confirm the structure.

### Materials

VS7 peptide is purchased from Hangzhou Xinbosi Biomedical.

HO-Dextran-CHO is purchased from Merck (Sigma-Aldrich).

### Method

HO-Dextran-CHO is conjugated to EGCG according to WO2006/124000 and WO2009/054813 to prepare HO-Dextran-EGCG.

VS7 is conjugated to HO-Dextran-EGCG via the conjugation between OH group on dextran and COOH group on VSP7 to form VS7-Dextran-EGCG (C′ linked). See FIG. 13.

Specifically, 1-1000 mg VS7 is incubated with 1-1000 mg HO-Dextran-EGCG, and 1-1000 mg N,N′-dicyclohexylcarbodiimide (DCC) in DMSO. The reaction is stirred at room temperature, prevented from light, under nitrogen for 24 hours. The reaction mixture is dialyzed (membrane Mw cutoff=2000 Da) against methanol and distilled water for 3 days. Next, the solution is freeze-dried to obtain lyophilized powder.

### Example 12: Efficacy Study of MINC-Anti-CD3, VS7-MINC-Anti-CD3, CST-MINC-Anti-CD3 or CLI-MINC-Anti-CD3 in an Acute Myocardial Infarction Mouse Model (Prophetic Example)

### Objective

This experiment is intended to demonstrate the therapeutic efficacy improvement of MINC-anti-CD3 compared to MINC-anti-CD3. Survival rate and histological analysis of heart infarction size are used to evaluate the therapeutic benefit of heart targeting peptide conjugated MINC-anti-CD3.

### Materials

Male C57BL/6 mice are purchase from BioLasco

AT-MINC-anti-CD3 is formulated following Example 4.

Anti-CD3 antibody is purchased from Biolegend.

MINC-anti-CD3 is prepared following Example 4.

### Method

Myocardial infarcts (MI) are generated in male C57BL/6-mice (10-12 weeks). Briefly, at day 1, after anesthesia with isoflurane, mice are intubated and connected to a small animal volume control ventilator. After exposing the heart via sternotomy, the left anterior descending artery (LAD) is permanently occluded with a suture. The sternum incision is closed and the mice are allowed to recover. 3-7 days post MI. At day 1 and day 2, the mice are divided into groups, receiving anti-CD3, MINC-anti-CD3, VS7-MINC-anti-CD3, CST-MINC-anti-CD3 or CLI-MINC-anti-CD3 at 5˜125 μg/mouse via tail vein injection, respectively. Survival rates are assessed up to 14 days after the surgery. At day 14, after the mice are anesthetized, Evans blue dye (0.1 g/ml) is injected into the abdominal aorta (1 ml). The heart is quickly removed, weighed and fixed with 4% paraformaldehyde. The infarct area is pale white, while the non-infarct area is red. Each slice is photographed and analyzed. The percentage of heart infarct size is calculated using computerized planimetry (Image J). The percentage of infarct size for each slice is calculated and then averaged across the slices.

LIST OF ABBREVIATIONS

| | |
|---|---|
| CVD | Cardiovascular Disease |
| MMP2 | Matrix Metalloproteinase 2 |
| AT1R | Angiotensin II Type 1 Receptor |
| CRIP2 | Cysteine-rich Protein 2 |
| VCAM-1 | Vascular Cell Adhesion Molecule 1 |
| ICAM-1 | Intercellular Adhesion Molecule 1 |
| IGF-1 | Insulin-like Growth Factor 1 |
| TGF-ß1 | Transforming Growth Factor Beta 1 |
| TIMP-1 | Tissue Inhibitor of Metalloproteinases 1 |
| VEGF | Vascular Endothelial Growth Factor |
| ß-FGF | Beta-Fibroblast Growth Factor |
| HGF | Hepatocyte Growth Factor |
| PDGF | Platelet-derived Growth Factor |
| IL-1 | Interleukin 1 |
| CCL-5 | Chemokine (C-C motif) Ligand 5 |
| TNF-α | Tumor Necrosis Factor Alpha |
| MCP-1 | Monocyte Chemoattractant Protein 1 |
| CXCR2 | C-X-C Chemokine Receptor Type 2 |
| MMP9 | Matrix Metalloproteinase 9 |
| PCSK9 | Proprotein Convertase Subtilisin/Kexin Type 9 |

The invention, and the manner and process of making and using it, are now described in such full, clear, concise, and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as the invention, the following claims conclude this specification.

What is claimed is:

1. A conjugate comprising:
(a) a cardiovascular disease (CVD)-targeting ligand, (b) a hydrophilic polymer of polyethylene glycol (PEG), polylactic acid (PLA), polylactic-co-glycolic acid (PLGA), or dextran, and (c) a flavonoid of EGCG, EC, EGC, or ECG, as shown in the structures below: 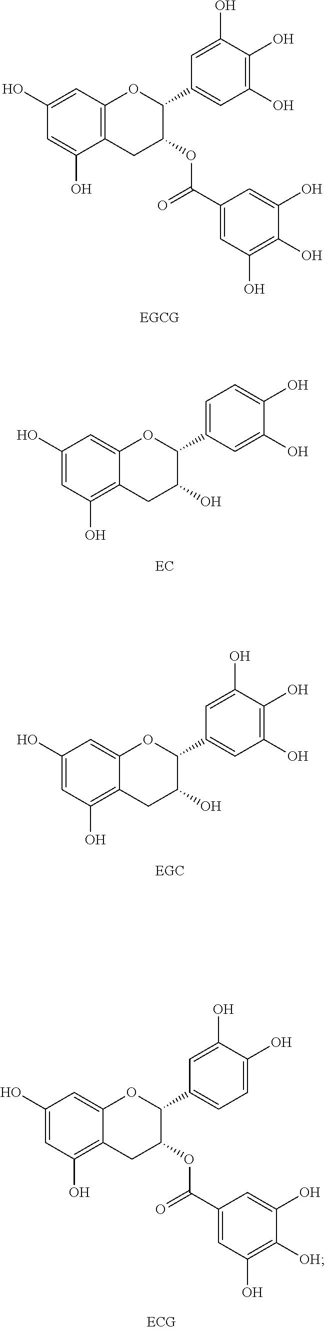 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
wherein the hydrophilic polymer covalently binds to the flavonoid and the CNS-targeting ligand,
wherein the CVD-targeting ligand is selected from the group consisting of:
VS7 peptide having the amino acid sequence of VVLVTSS (SEQ ID NO: 1),
CST peptide having the amino acid sequence of CSTSMLKAC (SEQ ID NO: 2),
CLI peptide having the amino acid sequence of CLIDLHVMC (SEQ ID NO: 3),
CTT peptide having the amino acid sequence of CTTHWGFTLC (SEQ ID NO: 4),
atrial natriuretic peptide (ANP) peptide having the amino acid sequence of SLRRSSCFGGRMDRIGAQSGLGCNSFRY (SEQ ID NO: 5),
CRS peptide having the amino acid sequence of CRSWNKADNRSC (SEQ ID NO: 6),
DF8 peptide having the amino acid sequence of DRVYIHPF (SEQ ID NO: 7),
CRP peptide having the amino acid sequence of CRPPR (SEQ ID NO: 8),
QV7 peptide having the amino acid sequence of QAQGQLV (SEQ ID NO: 9),
AV7 peptide having the amino acid sequence of ARRGQAV (SEQ ID NO: 10),
GV7 peptide having the amino acid sequence of GRRFIRV (SEQ ID NO: 11),
VR7 peptide having the amino acid sequence of VHPKQHR (SEQ ID NO: 12),
VK7 peptide having the amino acid sequence of VHSPNKK (SEQ ID NO: 13),
CC9 peptide having the amino acid sequence of CNNSKSHTC (SEQ ID NO: 14),
NA17 peptide having the amino acid sequence of NNQKIVNLKEKVAQLEA (SEQ ID NO: 15),
RGD peptide having the amino acid sequence of RGD,
GC11 peptide having the amino acid sequence of GPXRSGGGGKC (SEQ ID NO: 16),
KL9 peptide having the amino acid sequence of KKLVPRGSL (SEQ ID NO: 17),
CRK peptide having the amino acid sequence of CRKRLDRNC (SEQ ID NO: 18),
CRT peptide having the amino acid sequence of CRTLTVRKC (SEQ ID NO: 19),
CKR peptide having the amino acid sequence of CKRAVR (SEQ ID NO: 20),
CPK peptide having the amino acid sequence of CPKTRRVPC (SEQ ID NO: 21),
CAR peptide having the amino acid sequence of CARPAR (SEQ ID NO: 22),
CRS9 peptide having the amino acid sequence of CRSTRANPC (SEQ ID NO: 23),
GQ7 peptide having the amino acid sequence of GGGVFWQ (SEQ ID NO: 24),
HH7 peptide having the amino acid sequence of HGRVRPH (SEQ ID NO: 25),
CLH peptide having the amino acid sequence of CLHRGNSC (SEQ ID NO: 26), and
CRS12 peptide having the amino acid sequence of CRSWNKADNRSC (SEQ ID NO: 27).

2. The conjugate of claim 1, wherein the flavonoid is EGCG.

3. The conjugate of claim 1, wherein the hydrophilic polymer is PEG.

4. The conjugate of claim 1, wherein the flavonoid is EGCG and the hydrophilic polymer is PEG.

5. A nanoparticle composition comprising nanoparticles having: (a) an outer shell comprising the conjugate of claim 1, optionally (b) an inner shell comprising one or more flavonoid oligomer, and optionally (c) a drug encapsulated within the shells, wherein the flavonoid is EGCG, EC, EGC, or ECG, and the drug is effective to treat a cardiovascular disease.

6. The nanoparticle composition of claim 5, wherein the flavonoid in the conjugate is EGCG.

7. The nanoparticle composition of claim 5, wherein the hydrophilic polymer in the conjugate is PEG.

8. The nanoparticle composition of claim 5, wherein the flavonoid is EGCG and the hydrophilic polymer is PEG, in the conjugate.

9. The nanoparticle composition of claim 5, wherein the encapsulated drug is IGF-1, TGF-β1, MCP-1, TIMP-1, VEGF, β-FGF, endothelin-1, urocortin, VEGF, HGF, FGF, PDGF, TGF-β, neuregulin, NO-synthase, evasin-3, Evasin-4, IL-1 trap, IL-1RA, anti-CD3, anti-CD39, anti-CD73, anti-PD-1, anti-PD-L1, anti-PD-L2, anti-CTLA4, anti-GZM A, anti-GZM B, anti-IL-1β, anti-IL-8, anti-TNF-α, anti-CCL-5, anti-MCP-1, anti-CXCR2, anti-IL-6R, IL-19, anti-IL-12, anti-IL-23, anti-MMP9, anti-IL-1β, anti-TNF-α, anti-IL-1β, anti-IL-6, anti-IL-7, anti-IL-12 or anti-IL-23, sacubitrilsacubitril, valsartanvalsartan, aliskirenaliskiren, enoxaparinenoxaparin, clopidogrelclopidogrel, abciximababciximab, eptifibatideeptifibatide, bivalirudinbivalirudin, morphinemorphine, cyclosporine cyclosporine A, furosemidefurosemide, staphylokinasestaphylokinase, dapagliflozindapagliflozin, anakinraanakinra, canakinumabcanakinumab, rimonabantrimonabant, losmapimodlosmapimod, or darapladibdarapladib.

10. The nanoparticle composition of claim 5, wherein the outer shell further comprises a bare hydrophilic polymer-flavonoid conjugate that does not covalently bind to the CVD-targeting ligand.

11. A method for treating a CVD, comprising the step of administering to a subject in need thereof an effective amount of the nanoparticle composition of claim 5.

12. The method of claim 7, wherein the CVD disease is coronary artery diseases, myocardial infarction, heart failure, atherosclerosis, aneurysm, hypertensive heart disease, rheumatic heart disease, cardiomyopathy, abnormal heart rhythms, congenital heart disease, valvular heart disease, carditis, aortic aneurysms, peripheral artery disease, thromboembolic disease, or venous thrombosis.
